(19) United States
(12) Patent Application Publication
(10) Pub. No.: US 2015/0359456 A1
(43) Pub. Date: Dec. 17, 2015

(54) MEDICAL HOLDER DEVICE OR A FLEXIBLE MEDICAL TOOLTIP AND METHOD FOR CALCULATING THE POSITION OF THE TOOLTIP

(71) Applicant: BRAINLAB AG, Feldkirchen (DE)

(72) Inventors: Alexander URBAN, Forstinning (DE); Alexander DRUSE, Markt Schwaben (DE)

(73) Assignee: BRAINLAB AG, Feldkirchen (DE)

(21) Appl. No.: 14/761,434

(22) PCT Filed: Feb. 19, 2013
(86) PCT No.: PCT/EP2013/053221

§ 371 Date: Jul. 16, 2015

(51) Int. Cl. A61B 5/06

(52) U.S. Cl. CPC A61B 5/065

(57) ABSTRACT

A medical holder device to which a flexible medical tooltip comprising an optical fibre can exchangeably be attached, the medical holder device comprising an optical fibre connector for receiving the proximal end of the optical fibre, a light source adapted to emit light into the optical fibre, a light receiver adapted to receive light reflected at the distal end of the optical fibre and to output a corresponding output signal and a processing unit adapted to receive the output signal from the light receiver and to calculate a bending of the optical fibre.

The present invention relates to a medical holder device to which a flexible medical tooltip comprising an optical fibre can exchangeably be attached and a method for calculating the position of the distal end of the flexible tooltip comprising an optical fibre and being exchangeably attached to a medical holder device.

It is known in medical applications to use medical tooltips, such as needles for infusing a medical liquid, a biopsy needle or a stilet for supporting a structure such as a cannula. Since every tooltip is flexible to a certain degree, the tooltip can bend while it is used. The tooltip has a proximal end which is connected to a holder device and a distal end which is to be inserted into a body. In order to optimize the treatment, it is advantageous to know the position of the distal end of the tooltip. For this purpose, the distal end is typically tracked using an electromagnetic tracking system, wherein an electromagnetic transmitter is provided at the distal end of the tooltip. This document is directed to a different approach.

The present invention relates to a medical holder device to which a flexible medical tooltip comprising an optical fibre can exchangeably be attached. A medical holder device comprises an optical fibre connector for in particular releasably receiving the proximal end of the optical fibre, a light source adapted to emit light into the optical fibre, a light receiver adapted to receive light reflected at the distal end of the optical fibre and to output a corresponding output signal and a processing unit adapted to receive the output signal from the light receiver and to calculate a bending of the optical fibre from the output signal.

The medical holder device is a universal device to which a flexible medical tooltip can be attached. The flexible medical tooltip can be selected depending on the application. The tooltip can for example be a needle for supplying a medical liquid, a biopsy needle or a stilet. The optical fibre can be one of several components of the tooltip or the only component. In particular, the optical fibre can act as a stilet. If the optical fibre is one of a plurality of components of the tooltip, then the optical fibre may be placed on the inside or the outside of the tooltip. The optical fibre preferably has the same or a slightly, for example 5%, smaller length than the tooltip.

The optical fibre has a proximal end at the medical holder device and an opposite distal end. Preferably, the distal end is provided with a reflective coating for reflecting light which is coupled into the optical fibre at the proximal end. Via the optical fibre connector, the proximal end of the optical fibre is connected to the medical holder device.

In this document, the term “position” means the spatial location in up to three translational dimensions and/or the rotational alignment in up to three rotational dimensions. For the distal end of the tooltip, in most cases the spatial location is sufficient. If the optical fibre is only a part of the tooltip, then there is a known positional relation between the distal end of the optical fibre and the distal end of the tooltip. If the tooltip is not deformed by contact with an object, then the relative position of the distal end of the optical fibre or the tooltip to the medical holder device is referred to as neutral position.

The light source, which preferably is an LED, in particular a laser diode, emits light which is coupled into the optical fibre, for example via a coupling module. The coupling module can comprise a semi-transparent mirror. The light travels along the optical fibre towards the distal end, is reflected and returns to the proximal end of the optical fibre, where it is guided onto the light receiver, for example via the coupling module. The light receiver, such as a photo diode, outputs an output signal corresponding to the received light. The output signal can also be output data in which values corresponding to the received light are encoded.

The processing unit receives the output signal from the light receiver and calculates the bending of the optical fibre, for example based on the run time of the light in the optical fibre and/or the incident angle of the reflected light onto the light receiver. This is, for example, disclosed in the term paper “Navigationsverfahren in der Medizin” by Daniel Weber, summer term 2004, Universität Karlsruhe, Institut fair Rechnerentwurf and Fehlertoleranz, which is incorporated herein by reference, such that details of the calculation are not explained in detail in the present document.

The bending of the optical fibre means the direction and/or the amount of the deformation of the optical fibre. Preferably, it is assumed that the bending of the optical fibre, and therefore of the flexible medical tooltip, is equal over its length, such that the optical fibre has the shape of a circular arc. In this case, the deviation of the position of the distal end of the optical fibre can be calculated from the direction of the deformation, the length of the optical fibre and a parameter for describing the curvature of the optical fibre, such as the radius of the circular arc.

In one embodiment, the medical holder device further comprises a mechanical connector for holding the flexible tooltip. If the tooltip is comprised of the optical fibre only, then the mechanical connector fixes the proximal end of the optical fibre. If the tooltip comprises at least one other component other than the optical fibre, then the mechanical connector preferably fixes at least one of the other components. The mechanical connector might be a plug connector, a screw connector or any other suitable kind of connector.

Preferably, the mechanical connector includes the optical fibre connector to form a multi-functional connector. This means that, if the tooltip is attached to the medical holder device, the same multi-functional connector establishes both the mechanical connection between the medical holder device and the tooltip and the optical connection between the optical fibre and the light source and/or the light receiver of the medical holder device.

In one embodiment, the medical holder device further comprises an indication light source, wherein the processing unit is adapted to illuminate the indication light source if the bending of the optical fibre exceeds a predetermined threshold. The indication light source preferably is a light emitting diode (LED). In this embodiment, the indication light source indicates a warning if the distance between the distal end of the optical fibre, and therefore of the tooltip, when the tooltip is deformed and the neutral position exceeds a threshold. In this case, the location of the distal end of the tooltip deviates from the believed position.

In addition or as an alternative, the medical holder device further comprises means for illuminating an indication light source on the tooltip if the bending of the optical fibre exceeds a predetermined threshold. The effect is the same as in the previously described embodiment, but now the indication light source is located on the tooltip instead of on the medical holder device. It is to be noted that it is of course possible to provide one indication light source on the medical holder device and one indication light source on the tooltip.

The means for illuminating the indication light source on the tooltip can be a switch which opens and closes an electrical circuit which drives the indication light source. In this case, the multi-functional connector preferably provides an electrical connector for the electrical circuit. In an alternative, the means for illuminating the indication light source in the tooltip provides a control signal which opens and closes a switch on the tooltip, the switch opening and closing an electrical circuit for driving the indication light source. In this case, the means for illuminating an indication light source might be the processing unit.

It shall be noted that the indication light source can be replaced by any other warning notification means, such as an acoustic transducer for emitting an acoustic warning or a vibrator for emitting a tactile warning. The means for illuminating the indication light source can then be a means for driving the warning notification means.

In one embodiment, the medical holder device further comprises a marker device. With this marker device, the position of the medical holder device can preferably be determined in up to three rotational dimensions and/or up to three translational dimensions.

In another constellation, the medical holder device comprises only a single marker. This is sufficient in particular if the tooltip is guided using a guidance mechanism and the guidance mechanism is provided with a marker device. In this case, the position of the guidance system can be determined and the location of the distal end of the tooltip can be determined based on the relative position between the guidance system and the marker on the medical holder device. The guidance system allows a movement of the tooltip in one dimension only, preferably in a translational dimension.

It is the function of a marker to be detected by a marker detection device (for example, a camera or an ultrasound receiver or analytical devices, like CT or MRI), such that its spatial position (i.e. its spatial location and/or alignment) can be ascertained. The detection device is in particular part of a navigation system. The markers can be active markers. An active marker can for example emit electromagnetic radiation and/or waves, wherein said radiation can be in the infrared, visible and/or ultraviolet spectral range. The marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can also, however, exhibit a cornered for example, cubic shape.

A marker device can for example be a reference star or a pointer or one marker or more than one (individual) marker which are preferably in a predetermined spatial relationship. A marker device comprises one, two, three or more markers which are in case of two or more markers in a predetermined spatial relationship. This predetermined spatial relationship is in particular known to a navigation system and for example stored in a computer of the navigation system.

The present invention is also directed to a navigation system for computer-assisted surgery. This navigation system preferably comprises a computer for processing the data provided in accordance with the method as described in any one of the embodiments. The navigation system preferably comprises a detection device for detecting the position of the detection points which represent the main points and auxiliary points, in order to generate detection signals and to supply the generated detection signals to the computer such that the computer can determine the absolute main point data and absolute auxiliary point data on the basis of the detection signals received. In this way, the absolute point data can be provided to the computer. The navigation system also preferably comprises a user interface for receiving the calculation results from the computer (for example, the position of the main plane, the position of the auxiliary plane and/or the position of the standard plane). The user interface provides the received data to the user as information. Examples of a user interface include a monitor or a loudspeaker. The user interface can use any kind of indication signal (for example a visual signal, an audio signal and/or a vibration signal).

In one embodiment, a medical holder device further comprises a transmitter for transmitting the bending of the optical fibre and/or the position of the distal end of the tooltip relative to the medical holder device, the relative position being calculated by the possessing unit from the bending. The information is preferably transmitted to a navigation system which tracks the position of the medical holder device. If the bending of the optical fibre is transmitted, then the medical navigation system calculates the position of the distal end of the tooltip relative to the medical holder device from the bending. From the position of the medical holder device and the relative position, the navigation system can calculate the location of the distal end of the tooltip. This location can preferably be indicated in an image data set of a patient.

In one embodiment, the processing unit of the medical holder device is adapted to identify the tooltip attached to the medical holder device. As an option, the tooltip is identified by identification information provided by the tooltip, for example by an RFID chip of the tooltip. As another option, the tooltip can be identified by the shape of its connector for engaging the mechanical connector of the medical holder device. If the tooltip is identified, then the properties of the optical fibre are known to the processing unit. From the properties of the optical fibre and the run time and/or the incident angle of the reflected light onto the light receiver, the bending of the optical fibre can be calculated.

In one embodiment, the medical holder device comprises a housing and/or a coating, the housing and/or coating being sterilizable. In this case, the medical holder device can be used multiple times.

The present invention further relates to the system comprising a medical holder device as described above and a flexible medical tooltip attached to the medical holder device. The flexible medical tooltip is exchangeable and preferably disposable after one single use.

The present invention further relates to a method for calculating the position of the distal end of a flexible tooltip comprising an optical fibre and being exchangeably attached to a medical holder device. The method comprises the steps of emitting light into the proximal end of the optical fibre, receiving light reflected at the distal end of the optical fibre, calculating the bending of the optical fibre from the received reflected light and calculating the position of the distal end of the tooltip relative to the medical holder device from the bending. Details of the method are in analogy to the description of the medical holder device given above.

In one embodiment, the method further comprises the steps of determining the absolute position of the medical holder device and of calculating the absolute position of the distal end of the tooltip from the absolute position of the medical holder device and the bending of the optical fibre or the relative position of the distal end of the tooltip to the medical holder device. The absolute position of the distal end of the tooltip can then for example be indicated in an image data set of a patient and/or compared to a desired absolute position.

In one embodiment, the method further comprises the step of producing indication information if the bending exceeds the predetermined threshold. This means that a displacement of the distal end of the tooltip from its believed position can be determined and indicated.

In one embodiment, the method further comprises the step of providing the bending or the relative position of the distal end of the tooltip to a medical navigation system. With this information, the medical navigation system can track and/or navigate the distal end of the tooltip.

The method in accordance with the invention is in particular a data processing method. The data processing method is preferably performed using technical means, in particular a computer. The data processing method is in particular executed by or on the computer. The computer in particular comprises a processor and a memory in order to process the data, in particular electronically and/or optically. The calculating steps described are in particular performed by a computer. Determining steps or calculating steps are in particular steps of determining data within the framework of the technical data processing method, in particular within the framework of a program. A computer is in particular any kind of data processing device, in particular electronic data processing device. A computer can be a device which is generally thought of as such, for example desktop PCs, notebooks, netbooks, etc., but can also be any programmable apparatus, such as for example a mobile phone or an embedded processor. A computer can in particular comprise a system (network) of “sub-computers”, wherein each sub-computer represents a computer in its own right. The term “computer” includes a cloud computer, in particular a cloud server. The term “cloud computer” includes a cloud computer system which in particular comprises a system of at least one cloud computer and in particular a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing” which describes computation, software, data access and storage services which do not require the end user to know the physical location and/or configuration of the computer delivering a specific service. In particular, the term “cloud” is used as a metaphor for the internet (world wide web). In particular, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer in particular comprises interfaces in order to receive or output data and/or perform an analogue-to-digital conversion. The data are in particular data which represent physical properties and/or are generated from technical signals. The technical signals are in particular generated by means of (technical) detection devices (such as for example devices for detecting marker devices) and/or (technical) analytical devices (such as for example devices for performing imaging methods), wherein the technical signals are in particular electrical or optical signals. The technical signals in particular represent the data received or outputted by the computer.

The invention also relates to a program which, when running on a computer or when loaded onto a computer, causes the computer to perform one or more or all of the method steps described herein and/or to a program storage medium on which the program is stored (in particular in a non-transitory form) and/or to a computer on which the program is running or into the memory of which the program is loaded and/or to a signal wave, in particular a digital signal wave, carrying information which represents the program, in particular the aforementioned program, which in particular comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, in particular computer-readable data storage medium comprising computer-usable, in particular computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, in particular a data processing device comprising a digital processor (central processing unit or CPU) which executes the computer program elements and optionally a volatile memory (in particular, a random access memory or RAM) for storing data used for and/or produced by executing the computer program elements. Within the framework of the present invention, a computer-usable, in particular computer-readable data storage medium can be any data storage medium which can include, store, communicate, propagate or transport the program for use on or in connection with the instruction-executing system, apparatus or device. The computer-usable, in particular computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can in particular include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or vibration element incorporated into an instrument).

It lies within the scope of the present invention to combine one or more features of two or more embodiments as long as technically feasible in order to form a new embodiment.

The invention shall now be described with reference to an accompanying FIGURE which shows a medical navigation system and a system comprising a medical holder device with an attached flexible medical tooltip.

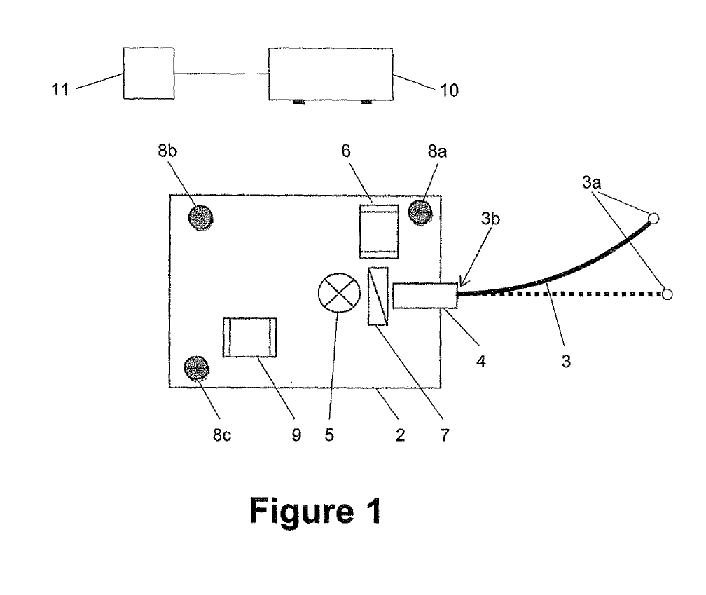

The only FIG. 1 shows a system 1 comprising a medical holder device 2 and a flexible medical tooltip 3. In this exemplary embodiment, the flexible medical tooltip 3 consists of an optical fibre only. The optical fibre 3 can be used as a stile for stabilizing a cannula.

The holder device 2 comprises a connector 4, an LED 5 as a light source, a light receiver 6, a coupling module 7, a marker device 8 comprising three markers 4, 8a, 8b and 8c and a processing unit 9.

A proximal end 3b of the optical fibre 3 is connected to the connector 4. The connector 4 is a multi-functional connector which mechanically and optically connects the optical fibre 3 to the medical holder device 2. With the mechanical connection, a movement of the holder device 2 also moves the optical fibre 3.

Light emitted from the LED 5 is coupled into the proximal end 3b of the optical fibre 3 through the coupling module 7 and the connector 4. In the present example, the coupling module 7 comprises a semi-transparent mirror which lets the light from the LED 5 pass into the connector 4, where it is coupled into the optical fibre 3. The light runs to the distal end 3a of the optical fibre 3, which comprises a reflective coating such that the light is reflected back towards the connector 4. The reflected light reaches the coupling module 7, where it is deflected onto the light receiver 6.

Without any external forces applied to the tooltip, the optical fibre 3 is typically straight as indicated by the dashed line, thus representing a neutral relative position between the distal end 3a of the optical fibre 3 and the medical holder device 2. If a force is applied to the optical fibre 3, a deformation of the optical fibre 3 occurs. In this exemplary embodiment, it is assumed that the deformation makes the optical fibre take the shape of a circular arc. This means that the position of the distal end 3a of the optical fibre relative to the holder device 2 deviates from the neutral position. An established registration between the distal end 3a of the optical fibre 3 and the holding device 2 thus becomes invalid.

The deformation or bending of the optical fibre 3 causes a characteristic pattern of the reflected light received by the light receiver 6. In particular, the incident angle of the reflected light onto the light receiver 6 depends on the curvature of the optical fibre 3. Depending on the incident light, the light receiver 6 generates an output signal which is provided to the processing unit 9. Based on the output signal of the light receiver 6, the processing unit 9 calculates the curvature of the optical fibre 3. Preferably, the processing unit 9 further calculates the direction of the curvature of the optical fibre 3, or more general of the flexible tooltip, preferably as an angle about a longitudinal axis of the connector 4 in which the proximal end 3b of the optical fibre 3 lies.

From the amount and direction of the bending of the optical fibre 3, the processing unit 9 calculates the position of the distal end 3a of the optical fibre 3 relative to the holder device 2.

A medical navigation system comprising a stereoscopic camera 10 and electronics 11, as known in the art, tracks the position of the holder device 2 by determining the spatial locations of the markers 8a, 8b and 8c. The processing unit 9 transmits the relative position between the distal end 3a of the optical fibre 3 and the holder device 2 to the electronics 11 of the navigation system. From the absolute position of the holder device 2, determined using the stereoscopic camera 10 and the marker device 8, and the relative position between the distal end 3a of the optical fibre and the holder device 2, the electronics 11 of the navigation system can calculate the absolute position of the distal end 3a of the optical fibre 3. The distal end 3a can therefore be tracked.

The displacement of the distal end 3a of the optical fibre 3 depends on the curvature and the length of the optical fibre 3. The length of the optical fibre 3 can be determined from the run time of a light pulse generated by the LED 5 and received by the light receiver 6. In addition or as an alternative, the length of the optical fibre 3 can be determined by identifying the optical fibre 3 and determining the length corresponding to the identification, for example from a database.

It might not be necessary to track the position of the distal end 3a of the optical fibre 3. However, a deviation of the position of the distal end 3a of the optical fibre 3 from its neutral position relative to the holder device 2 should be prevented. In this case, the processing unit 9 calculates the displacement of the distal end 3a of the optical fibre from its neutral position from the bending of the optical fibre 3 represented by the output signal of the light receiver 6. If the displacement exceeds a predetermined threshold, the processing unit 9 issues a warning, such as an optical warning by illuminating a light source, an acoustical warning by driving an acoustic transducer to make a sound, or a tactile warning by making the holder device 2 vibrate.

1. A medical holder device to which a flexible medical tooltip comprising an optical fiber can exchangeably be attached, the medical holder device comprising an optical fibre connector for receiving the proximal end of the optical fiber, a light source adapted to emit light into the optical fiber, a light receiver adapted to receive light reflected at the distal end of the optical fiber and to output a corresponding output signal and a processing unit adapted to receive the output signal from the light receiver and to calculate a bending of the optical fiber, wherein the medical holder device further comprises a marker device.

2. The medical holder device according to claim 1, further comprising a mechanical connector for holding the flexible tooltip.

3. The medical holder device according to claim 2, wherein the mechanical connector includes the optical fiber connector to form a multi-functional connector.

4. The medical holder device according to claim 1, further comprising an indication light source, wherein the processing unit is adapted to illuminate the indication light source if the bending of the optical fiber exceeds a predetermined threshold.

5. The medical holder device according to claim 1, further comprising an illumination member for illuminating an indication light source on the tooltip if the bending of the optical fiber exceeds a predetermined threshold.

6. (canceled)

7. The medical holder device according to claim 1, further comprising a transmitter for transmitting the bending of the optical fiber and/or the position of the distal end of the tooltip relative to the medical holder device, the relative position being calculated by the processing unit from the bending.

8. The medical holder device according to claim 1, wherein the processing unit is adapted to identify the tooltip attached to the medical holder device.

9. The medical holder device according to claim 1, comprising a housing and/or a coating, the housing and/or coating being sterilisable.

10. A system comprising a medical holder device according to claim 1 and a flexible medical tooltip attached to the medical holder device.

11. A computer-implemented data processing method for calculating the position of a distal end of a flexible tooltip comprising an optical fiber and being exchangeably attached with a medical holder device, comprising executing, on a processor of a computer, the steps of causing the emission of light into the proximal end of the optical fiber, causing to receive light reflected at the distal end of the optical fiber, calculating the bending of the optical fiber from the received reflected light calculating a position of the distal end of the tooltip relative to the medical holder device from the bending and providing the relative position of the distal end of the tooltip to a medical navigation system.

12. The method according to claim 11, further comprising executing, on the processor of the computer, the step of calculating the absolute position of the distal end of the tooltip from the absolute position of the medical holder device and the bending of the optical fiber or the relative position of the distal end of the tooltip to the medical holder device.

13. The method according to claim 11, further comprising executing, on the processor of the computer, the step of producing an indication information if the bending exceeds a predetermined threshold.

14. The method according to claim 13, further comprising executing, on the processor of the computer, the step of providing a bending or the relative position of the distal end of the tooltip to the medical navigation system.

15. A non-transitory computer-readable storage medium storing a computer program which, when running on a processor of a computer causes the computer to perform a data processing method for calculating the position of a distal end of a flexible tooltip comprising an optical fiber and being exchangeably attached to a medical holder device, comprising the steps of:
causing, by the processor, the emission of light into the proximal end of the optical fiber, causing, by the processor, to receive light reflected at the distal end of the optical fiber;
calculating, by the processor, the bending of the optical fiber from the received reflected light;
calculating, by the processor, a position of the distal end of the tooltip relative to the medical holder device from the bending; and
providing, by the processor, the relative position of the distal end of the tooltip to a medical navigation system.

16. A computer comprising the non-transitory computer-readable storage medium according to claim 15.
